(19) United States
(12) Patent Application Publication
(10) Pub. No.: US 2024/0296961 A1
(43) Pub. Date: Sep. 5, 2024

(54) SYSTEM AND METHOD FOR MODELING NEURONAL SYNAPTIC FUNCTIONALITY ON A COMBINED SOFTWARE AND HARDWARE ARCHITECTURE

(71) Applicant: The Research Foundation for the State University of New York, Albany, NY (US)

(72) Inventors: Janet L Paluh, Albany, NY (US); Rounak Chatterjee, Kolkata (IN); Soham Mondal, Bengaluru Karnataka (IN); Souradeep Chowdhury, Kolkata (IN); Arnab Raha, Santa Clara, CA (US); Amitava Mukherjee, Kolkata (IN)

(21) Appl. No.: 18/569,431

(22) PCT Filed: Jun. 13, 2022
(86) PCT No.: PCT/US2022/033185

§ 371 Date: Dec. 12, 2023

(30) Foreign Application Priority Data
Jun. 12, 2021 (IN) 202111026213

(51) Int. Cl. G16H 50/50; G06N 3/049; G06N 3/063

(52) U.S. Cl. CPC G16H 50/50; G06N 3/049; G06N 3/063

(57) ABSTRACT

A system and method for modeling neuronal synaptic functionality at least partially instantiated on an optimized computation core of one or more high-speed processors. The synaptic model is preferably created as a neural net and includes, at least, a presynaptic component with a presynaptic target having, at least, a plasticity parameter and activity spike strength. The model also includes a retrograde signaling component with a retrograde messenger that selectively generates a molecular uptake signal, and a postsynaptic receptor component. The retrograde messenger acts on a presynaptic target to modulate the plasticity parameter and activity spike strength of the presynaptic component based upon a calculated molecular uptake at the postsynaptic receptor component to generate the molecular uptake signal which is then transmitted to the presynaptic component.

### BACKGROUND OF THE INVENTION

### 1. Field of the Invention

The present invention generally relates to computer software and hardware that can generate biological models. More particularly, the present invention relates to a system and method for generating an accurate biological model of a neuronal synapse for purposed of medical diagnoses and predictive drug interactions.

### 2. Description of the Related Art

Neural signaling within organic creatures creates memory and control bodily functions by conversion of an electrical signal into the inter-neuronal transmission of chemical neurotransmitter information at neuronal synapses. At a synapse, one neuron sends a message to a target neuron to communicate the electrical signal. The events are non-spontaneous and dependent on input intensity and frequency, and the measured electrophysiological response is an action potential that brings into play the complex physiology of neuron dendritic, cell body, axonal hillock and axonal compartments. The largest contribution of information passing in neural circuits occurs at synapses and is regulated by a diversity of synaptic plasticity mechanisms that must operate over a finite timescale. Long-term changes impact learning and memory and short-term changes support synaptic computations.

These timescale changes can be described in terms of spike timing dependent plasticity (STDP) that relates changes in synaptic strength, or synaptic weights, to the timing of presynaptic and postsynaptic spikes, which is a key mechanism in memory formation. When linked to synaptic stability, this plasticity can be used to describe flexible or stable memories and complex topologies of neural networks.

Information transfer between neurons from neurotransmitter release is a highly regulated, yet probabilistic process. In short term plasticity to modify neuronal circuits, synapses are viewed as active filters of information, not just conveyers, reducing noise and enhancing relevant information. A known mechanism to dynamically fine tune the probability of neurotransmitter release is through local feedback regulation. Intermediate initial release dynamics behave as band pass filters. This allows adaptive regulation of changes in network activity and enables neurons to respond to prolonged alterations.

However, relative changes to this homeostatic mechanism arising from synaptopathies, that is physiological defects and errors in the synapse itself, affect synaptic plasticity has not been thoroughly examined. One example of a synaptic system that is not well examined is the glutamatergic synapse, which is a low-pass filter type. Glutamatergic neurons encompass most of the synapses in the human central nervous system (CNS) and are known to be relevant to cognitive decline.

Spiking Neural Networks (SNNs) are used to model synapses to reflect action potential spikes and a large number of SNN software and hardware models have been proposed for mimicking neurological behavior. Although providing general biological accuracy, these software-based simulations encounter extraordinarily high computational costs for subsequent hardware development while performing numerical simulations. Hence, modern computers often fail to obtain real-time performance when scaled to simulate large neural networks. For example, a one second simulation of a network composed of 8 million neurons that includes 4 billion integrate-and-fire synapses when analyzed on the IBM® Blue Gene® supercomputer using 2048 processors, takes approximately 80 minutes.

Attempts have been made to optimize biological models, such modeling neural signaling, by co-development of software and hardware implementation jointly, with considerations of compactness, power-efficiency and ease of implementation of circuits. In this regard, circuits involving feedback mechanisms have included analog circuit designs of SNNs. The relative efficiency of an analog versus digital implementation varies dependent on the required signal-to-noise ratio. In neural dynamics, challenging temporal features are present, such as high and nonuniform pulse latency and activity dependent synaptic plasticity that results in long-lasting long-term potentiation (LTP) processing. This creates a need for capacitors of value greater than 0.1 F, which imposes physical constraints. This challenge is significant and nullifies much of the advantage offered by analog circuits in terms of a smaller number of transistors.

Analog circuits also suffer from transistor mismatch, process variation and model size limitations owing to a process termed “gate fan-out” that relates the number of gate inputs to a single original logic gate. Another challenge is in reliable analog memory which has not yet been robustly achieved in regard to storage of significant processing values. This includes synaptic weight whose subtle variations have significant importance to the accuracy of the entire system.

A number of synaptic models have been developed for digital implementation including the Time Machine approach, SpinNaker, Neurogrid, and BrainScales, among others. BrainScales is a wafer-scale neuromorphic system, in which each wafer contains reticles with eight High-Count Analog Neural Network (HiCANN) dice. Each such HiCANN die has the capability to emulate 512 adaptive exponential neuron models. SpinNaker is a one-million-core supercomputer, developed exclusively for massively-parallel real-time simulation of large-scale neural networks, making it one of the largest digital neuromorphic platforms to date. Neurogrid is a real-time system consisting of over one million quadratic integrate-and-fire neurons, in which neuron and synapse dynamics are emulated using analog circuits and communications are performed by digital means, for simulation of over a billion synapses. However, since these models have been developed with the intention of utilization in Spiking Neural Network (SNN) architectures, they still only approximate the effect of the numerous parameters in a neuron.

In current large scale neuromorphic platforms that rely on SNN architectures, the exclusive abstraction of intracellular dynamics of a neuron that ignores other biologically modulated parameters is severely restricted in applications to understand and modulate plasticity. For example, glutamate is known to be the main excitatory neurotransmitter in the CNS and accounts for 90% of the total neurotransmitter usage in the CNS. Glutamate and glutamatergic neurotransmission dysfunction is a central mechanism in Autism Spectrum Disorder (ASD) and has broader impacts on neurodegeneration, as well as on psychiatric disorders. Thus, extant modeling lacks sufficient resolution and accuracy in this respect.

Moreover, less investigated in regard to the synaptic involvement in learning and memory is the relative impact of altered biological mechanisms on synaptic strength, owing to lack of generalized computational models as well as scalable hardware architectures that can process such complexity. Therefore, a computational neuronal model is needed which is adapted to study the implications to synaptic strength and efficiency when stochastic variations occur in underlying mechanisms, and which can also be implemented into hardware to optimize the accuracy, efficiency and speed of the model.

### BRIEF SUMMARY OF THE INVENTION

Briefly described, the present invention is for a system and method for modeling neuronal synaptic functionality at least partially on an optimized computation core of one or more high-speed processors. The synaptic model can be instantiated in software, hardware or both, but preferably uses the optimized logic provided by the computation core to improve processing speed to operate up to real time. The synaptic model includes, at least, a presynaptic component with a presynaptic target having, at least, a plasticity parameter and activity spike strength. The model also includes a retrograde signaling component with a retrograde messenger that selectively generates a molecular uptake signal, and a postsynaptic receptor component. The retrograde messenger acts on a presynaptic target to modulate the plasticity parameter and activity spike strength of the presynaptic component based upon a calculated molecular uptake at the postsynaptic receptor component to generate the molecular uptake signal and transmit it back to the presynaptic receptor component.

In one embodiment, the system for modeling neuronal synaptic functionality includes the computation core comprised of one or more high-speed processors, and the synaptic model. The synaptic model is comprised of, at least, a presynaptic component including at least a presynaptic target neuron having, at least, a plasticity parameter and activity spike strength. The synaptic model further includes a retrograde signaling component including a retrograde messenger that selectively generates a molecular uptake signal, and a postsynaptic receptor component. The retrograde messenger acts on a presynaptic target to modulate the plasticity parameter and activity spike strength of the presynaptic component based upon a calculated molecular uptake at the postsynaptic receptor component, the retrograde messenger thereby generating the molecular uptake signal and transmitting it back to the presynaptic receptor component.

The molecular uptake signal of the retrograde signaling component can be modulatable in both duration and latency, and the retrograde signaling component can further determine an uptake and degradation of molecules at the postsynaptic receptor component and modulating the molecular uptake signal based upon such determination. Further, the retrograde messenger can be located at the postsynaptic component and losslessly transmits the molecular uptake signal to the presynaptic component.

The postsynaptic receptor components can include, at least, models for ionotropic glutamate receptors (iGluR) and metabotropic glutamate receptors (mGluR). The synaptic model can also model calcium-mediated synaptic events, and can further models synaptic strength as influenced by presynaptic and postsynaptic activity in an activity-dependent synaptic plasticity process.

The computation core, in one embodiment, is configured to work with an IEEE-754 single precision floating point number system. The computation core can further be pipelined for a mantissa operation.

In one embodiment, the invention includes a method of modeling neuronal synaptic functionality including the steps of configuring a synaptic model on a computation core comprised of one or more high-speed processors, where the synaptic model is created from the steps of configuring a presynaptic component including at least a presynaptic target having, at least, a plasticity parameter and activity spike strength, configuring a retrograde signaling component including a retrograde messenger, selectively generating a molecular uptake signal from the retrograde messenger, and configuring a postsynaptic receptor component. The method continues by modeling a presynaptic target at the retrograde messenger by modulating the plasticity parameter and activity spike strength of the presynaptic component based upon a calculated molecular uptake at the postsynaptic receptor component and transmitting the molecular uptake signal to the presynaptic component.

The present invention advantageously utilizes a retrograde messenger mediated plasticity (RMMP) model, as opposed to a glial cell-mediated model, to capture the speed of the hardware/software network. The model can therefore allow large-scale simulation of neural networks.

In one embodiment, the system and method model a RMMP bipartite neuron system in which there are no intermediary dynamics considered in the synaptic cleft, and hence the synaptic current from the presynaptic region is the same as the synaptic current entering the postsynaptic region. The dynamics of retrograde messengers that travel across the synapse via diffusion are the feedback initiator in the model. This allows the expanse of the biological detail in a digital framework to address multiple pre- and postsynaptic interacting molecular mechanisms found in synaptopathies, and the cross-evaluation dysfunction by a rapid multi-classification comparison. Expanded model detail can include receptor inhibitors and activators, including allosteric regulation, as well as receptor type ratios, synaptic cell adhesion molecules, and calcium signaling and organelle stores.

One advantage of the present invention is that it allows a diverse and highly adaptable design of neuron synapse function and dysfunction in software and hardware, utilizing the hardware as a digital logic accelerator. The system can therefore reproduce complex neuropathologies that are synaptopathies which have a foundation in molecular biomechanisms and allows comparative analysis of the synaptic dysfunction mechanisms as well as analysis of drug activity on synaptic function.

The system and method can define numerous parameters in neurons to investigate synaptic strength and efficiency of neuron spiking in synaptopathies with minimal error and no loss in speed of processivity, and provides the ability to examine individual and combined impacts of synaptopathies on synaptic plasticity in real time. The system and method therefore allow the incorporation of feedback regulation to calculate high-resolution neurotransmitter outcomes.

Another advantage of the system and method is that through the use of at least partial implementation of the synaptic model in hardware, it can be optimized for area compactness and high performance. It can therefore be integrable and interface with extant health platforms for evaluation of diagnosis, and can also interface with prosthetic interfaces in the central nervous system.

While the system and method can be implemented purely in software on a generic hardware platform, the modeling of at least partial digital logic in hardware provides performance in a dynamic timeframe reducing effective runtime for investigations interested in results at irregular intervals. This design methodology can also optimize quality energy tradeoffs in hardware based biomechanism models on different precision types such as int, fp, posit, etc. The system and method can reduce the overall computational cost to produce high fidelity numerical outputs by leveraging a scalable real time performance-optimized hardware design. The design methodology can further incorporate techniques to implement high-resolution biomechanism models with low computational area and low power consumption by emulating neuron spiking with minimum possible error without sacrificing speed of processivity of the digital system implemented as hardware.

### BRIEF DESCRIPTION OF THE DRAWINGS

FIG. 1 is an illustrative diagram of one embodiment of the neuron system model utilized in the present invention, illustrating the presynaptic region, the retrograde messenger, and postsynaptic region.

FIG. 2 is a diagram of one embodiment of a hardware circuit implementing digital logic for at least partial instantiation of the synaptic model.

FIG. 3 is an object diagram illustrating the objects comprising one embodiment of the software architecture of the present invention.

FIG. 4 is a diagram of three arithmetic submodules used in the present software architecture, with each task representing the number of pipelined stages involved for that task in the hardware architecture.

FIG. 5 is a representational diagram of the representation of affected components and modulators in the model for each considered Autism Spectrum Disorder (ASD) pathway scenario.

FIG. 6 is a classification diagram of ASD mechanisms considered based on distortion from the ideal synaptic output potential, with ideal distortion response set at 1.

FIG. 7 is a diagram of graphs demonstration the effect of allosteric modulation in tuberous sclerosis complex (TSC).

### DETAILED DESCRIPTION OF THE INVENTION

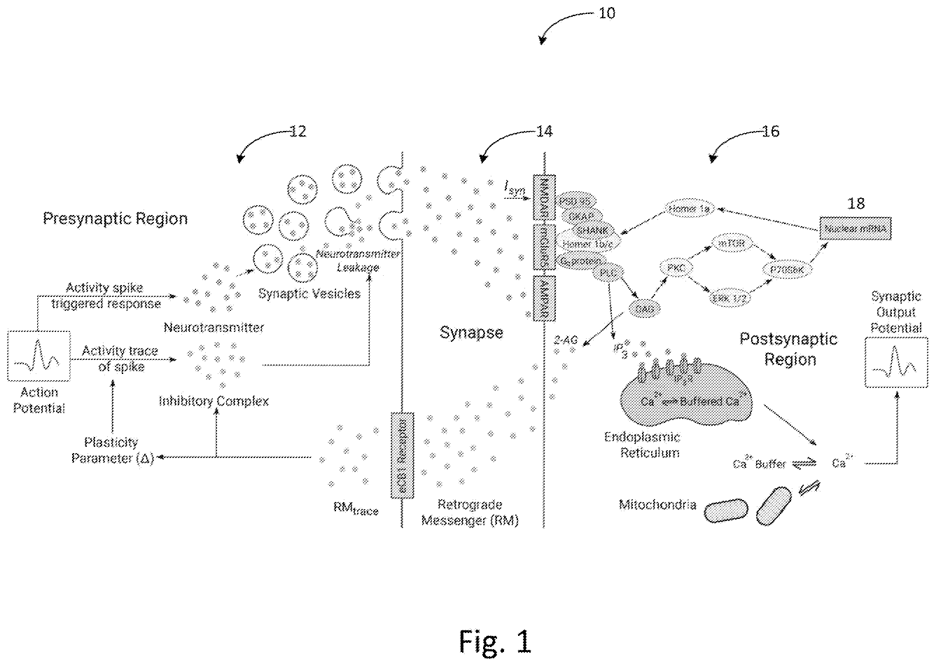

With reference to the figures in which like numerals represent like elements throughout the several views, FIG. 1 is an illustrative diagram of one embodiment of the neuron system model 10 utilized in the present invention, illustrating the presynaptic region 12, the retrograde messenger 14, and postsynaptic region 16. The presynaptic region 12 and postsynaptic region 16 dynamics are described for the model. The synaptic cleft acts as a lossless channel and is not involved in any dynamic processes involving neuronal communication. Proteins expressed in yellow are involved in the interactions between the locally transcribed nuclear mRNA 18 and the synaptic gate receptors as indicated by dotted arrows. Proteins represented in pink are bridge proteins involved in inter-receptor dynamics. The parameters involved in synaptic activity are linked by solid arrows. The roles of the proteins in synaptic activity diagrammed here are described in the text in regard to ASD mechanisms considered herein.

In FIG. 1, the main elements of the synaptic dynamics model are illustrated. Two primary components in our software for synapse dynamics are retrograde signaling at the retrograde messenger 14 and postsynaptic receptor type at postsynaptic region 16. Retrograde signaling systems integrate the postsynaptic response with regulation of presynaptic output generating rapid changes in synaptic strength. Although diverse chemical messengers exist, retrograde models share many basic steps. Firstly, the production and release of retrograde messengers from postsynaptic cells are regulated by postsynaptic calcium and triggered by postsynaptic metabotropic receptors and second messenger. Secondly, the entirety of the concentration of retrograde messenger 14 produced at the postsynaptic region is assumed to be transmitted to the presynaptic receptors with no losses in transmission or within the synaptic cavity. While such an assumption might feel out of place, we consider that such losses if they happen initially would eventually saturate to a point in which no loss occurs. The retrograde messenger 14 acts on the presynaptic target of the presynaptic region 12 to modulate the plasticity parameter and activity spike strength in the presynaptic region 12.

The duration and latency of a retrograde signal is controlled by parameters of uptake and degradation of the molecules at a cellular level. Synaptic signaling synapses feature a re-uptake mechanism at the presynaptic neuron, which provides a means for removing neurotransmitters from the synaptic cleft and for recycling them for future reuse. As a result, the nonlinear relationship between the release of a retrograde molecular uptake signal at the presynaptic region 12 and the magnitude and duration of the retrograde signal available to activate presynaptic receptors must be considered in the ultimate effect on synaptic strength. A final consideration is the extent to which the release of the retrograde messenger 14 can be sustained. Many cells contain few vesicles and dense core secretory granules in their dendrites, which suggest that it would be possible to deplete the release of conventional neurotransmitters, peptides, and growth factors. The low density of vesicles suggests that the dendritic release may be much more prone to depletion. The ability to recover from depletion would then depend crucially on endocytosis and vesicle and granule refilling. Alternately, some lipid-derived messengers are produced on, and as a result the release of these messengers may be sustained. To describe the regulated release of neurotransmitter at the synapse, one can consider the lipophilic (lipid-derived) retrograde signaling model.

The output of the model for observing effects of plasticity is calcium dynamics in the presynaptic region 12 and therefore can determine the impact of all the processes in the model that impact that parameter. Plasticity is achieved in the system by increasing the latency of calcium signals, which in turn can lower the peak calcium levels. Activation of Gq-coupled receptors such as mGluRI in the postsynaptic region 12 can promote release of inhibitory factors in the presynaptic region by increasing the production of retrograde messenger 14 that in turn reduces calcium levels.

The present invention therefore provides a biologically descriptive synaptic model for neural communication, with primary focus on modelling the complexity of a synapse in a way that can translate into, at least, several competent hardware models. The system can itself be instantiated as a neural net architecture. Large scale implementation of such models is aimed at utility in designing biologically extensive neuronal circuits. We focus on Autism Spectrum Disorders in which a variety of synaptopathy mechanisms operate in various syndromes. By generating a set of ASD interrogation algorithms (ASDint) one can model changes to synaptic dynamics in regard to corresponding disease variables and observe the spread of their impact in larger neural circuits. Development of ASDint core algorithm provides a powerful new computational and hardware approach to benefit ASD experimental analysis and to help predict manifestation of symptoms for ASD. This is accomplished by abstraction of neuronal behavior at the synapse while describing the complexity involved in the interactions of the implicated primary state variables. Importantly, such an approach is not restricted to ASD and the present algorithmic core can be used to design network models for any such synaptopathies with complex dynamics.

Most of the synapses in the CNS are low pass filters. While the synaptic communication properties are not ideal, the attenuation is not significant. For a large network of neurons consisting of many synaptic nodes, it is important that the signal amplitude is not attenuated to a large degree or else distant synaptic nodes in the neural communication framework will not be able to communicate with each other. Synapses modulate not only the amplitude of the action potential but also the selectivity and accuracy of the synaptic output response. The filter form of synapses has decisive implications on their noise response.

Synapses as such can be widely classified into three such classes: high pass, low pass and band pass filters. Those synapses with release probability below 0.3 act as high pass filters. Such synapses have very high selectivity and noise resistance to environmental procedures. However, the attenuation to synaptic output potential is also very significant in such synapses. Synapses with initial probability of neurotransmitter release greater than 0.7 act as low pass filters. Such synapses have very low selectivity, while attenuation to synaptic output potential is also rather insignificant in such synapses. However, the resulting synaptic transmission is affected by noise generated by environmental procedures to an observable degree. Synapses with intermediate initial release probability, between 0.3 and 0.7, act as band pass filters. In the models described herein, the initial neurotransmitter release probability of 0.5 is used, which adequately describes a low pass filter glutamatergic synapse.

Among the six main neurotransmitters in the human body, the main excitatory neurotransmitter in the brain is glutamate, which activates several postsynaptic receptors. Two primary types of receptors encountered in a glutamatergic neuron are ionotropic Glutamate Receptors (iGluR) and metabotropic Glutamate Receptors (mGluR). The mGluRs bind glutamate within a large extracellular domain and transmit signals through the receptor protein to intracellular secondary messenger signals. The receptors primarily involved in this process are the group I mGluRs: mGluRI and mGluR5. Ionotropic glutamate receptors (iGluRs) are faster responding and the two major types of iGluRs: a-amino-3-hydroxy-5-methyl-4-isoxazolepropionic acid (AMPA) and N-methyl D-aspartate (NMDA) have central roles in hippocampal synaptic plasticity. Both are ligand-gated ion channels and have unique properties that subserve different phases of synaptic plasticity. Glutamate released from the presynaptic neuron opens AMPA receptors to depolarize the postsynaptic cell. Each AMPA receptor has four sites to which an agonist (such as glutamate) can bind, one for each subunit. The channel opens when two sites become occupied, and current increases with subsequent binding. The AMPA receptor's permeability to calcium, and other cations such as sodium and potassium, is governed by the presence of the GluA2 subunit in the AMPARs. The presence of GluA2 renders the channel impermeable to calcium and is believed to guard against excitotoxicity.

Calcium-mediated synaptic events are believed to sustain temporary holding of information as in working memory and is preferably considered in the described models herein. To enable the flow of synaptic current through the postsynaptic receptors, mGluRI-type receptors coupled to the G protein (Gq) are activated. Together calcium and Gq activate phospholipase C beta (PLC), which cleaves the lipid phosphatidylinositol bisphosphate (PIP2) into diacylglycerol (DAG) and inositol trisphosphate (IP3). DAG is converted into the endocannabinoid 2-arachidonoylglycerol (2-AG). The rate-limiting and Ca2+-sensitive step in 2-AG production is the formation DAG. IP3 and DAG are free to diffuse through the cell cytoplasm and their impacts can be described computationally

In the described models, the role of mitochondria and the endoplasmic reticulum (ER) in calcium dynamics are considered. Mitochondria are important for numerous roles related to synaptic transmission and neurodegeneration including calcium regulation in neurons. Calcium mobilization from mitochondria is controlled by neurotransmitter release as well as more complex proposed buffering roles.

When IP3 binds to an IP3 receptor (IPR) on the ER membrane it causes the release of Ca2+ from the ER. Five pathways have been considered in the modulation of Ca2+ influx, which we describe in a pair of dynamic equations. Thus, we efficiently incorporate abstractions of several terms in our model (FIG. 1) to describe these dynamics. Ca2+ taken up into (Juni), or released from (Jmito) mitochondria, and that bound to (Jon), or released from (Joff), Ca2+ buffers are considered to create a constant Ca2+ flow that does not vary with instigation of cell membrane receptors. 2-AG then regulates presynaptic pathways to affect synaptic transmission by decreasing the probability of neurotransmitter release from the presynaptic terminal. In the model, the common cell structures act as an impedance and the calcium buffers in the cytoplasm provide capacitance and are not a part of the signaling circuit, hence acting as a channel to drain higher frequencies away.

Synaptic strength is influenced by pre- and postsynaptic activity in activity-dependent synaptic plasticity processes such as long-term potentiation (LTP) and long-term depression (LTD). Hebbian plasticity is used to define these features in synaptic plasticity. The N-methyl-D-aspartate receptors (NMDARs) are calcium permeable and when activated, allow an influx of calcium needed for the induction of LTP. However, NMDARs require both presynaptic transmitter release and postsynaptic depolarization for activation. Enhancement in the amplitude of action potential takes place when both the presynaptic and postsynaptic regions are active, resulting in potentiation of synaptic output potential. However, when either region is selectively active, the amplitude of action potential is attenuated, resulting in depression of synaptic output potential. These two processes together constitute the synaptic plasticity mechanism and hence act as substrates for fundamental brain function. LTP is a process involving such persistent enhancement of synaptic gain resulting in a long-lasting increase in synaptic transmission gain between the neurons. It is an important process in the context of synaptic plasticity. LTP recording is widely considered to be the cellular model for storage of information in the brain. LTD is an opposite process that modulates and controls the effect of LTP in the brain. Full opening of the NMDAR channel and the consequent influx of calcium requires both the binding of glutamate to the receptor and postsynaptic depolarization.

The induction of potentiation is dependent on activation of NMDARs and a rise in postsynaptic calcium. The NMDAR dependence provides a ready explanation for the associativity and asymmetry of Hebbian learning rule. The binding of glutamate follows the release of a transmitter by the presynaptic spike, and the postsynaptic depolarization is provided by the postsynaptic spike. Thus, neither the release of glutamate alone nor the postsynaptic spike alone will result in the opening of the receptor. Both must occur at the same time. In the frog optic tectum and in cultured hippocampal cells no potentiation was observed when the presynaptic spike preceded the postsynaptic spike by more than 20 ms and no depression was observed when the presynaptic spike followed the postsynaptic spike by more than 20 ms. LTD is induced at a lower concentration of calcium than required for induction of LTP, however the parameters of the timing window for depression are not fully predicted by the expected calcium concentration alone. In layer V/VI of the neocortex of the developing frog optic tectum and cultured hippocampal cells, the depression, like the potentiation, depended on the activation of NMDARs, but the depression found in layer II/III pyramidal cells of the somatosensory cortex did not. It is observed for layer II/III pyramidal cells that the interval for depression is considerably larger than the interval for potentiation. All of these learning rules are asymmetric in that positive actions have different effects than negative delays.

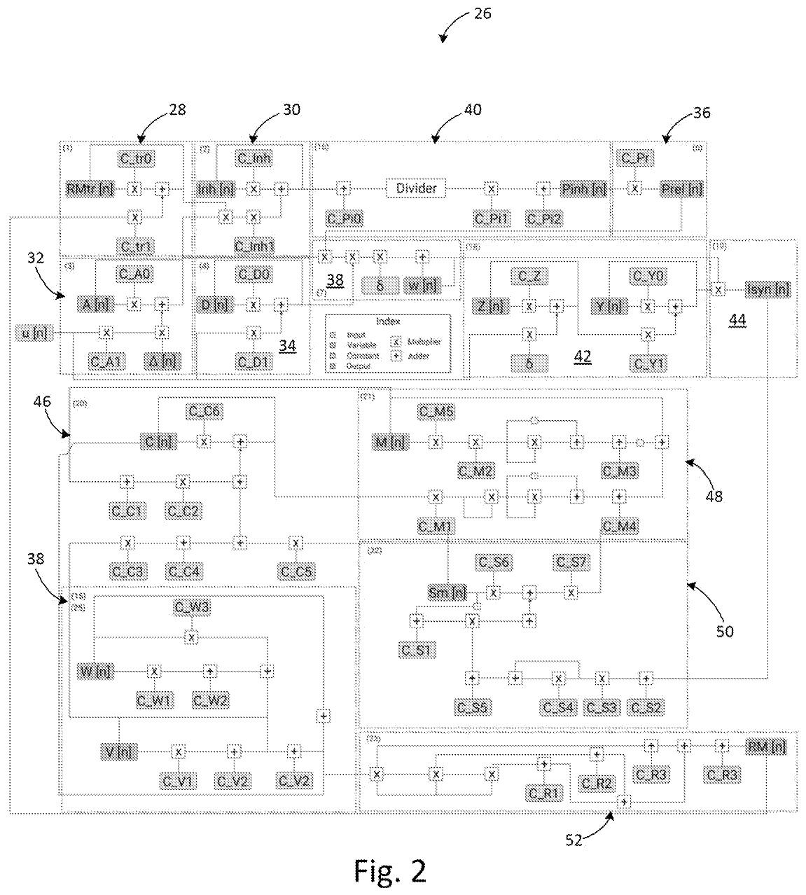

Accordingly, in one embodiment, the presynaptic region 12, in the model shown in FIG. 1, a logical presynaptic component can be constructed using a neuronal model which accounts for the processes involved in synaptic plasticity in a synapse mediated by retrograde messengers (RMs), such as retrograde messenger 14. The model, described in digital logic FIG. 2, demonstrates the synaptic current release from the presynaptic region when activity spike and retrograde messenger are provided to it. The amount of RM that diffuses into the presynaptic neuron consists of RMtrace. Activity trace of input spike considers the effect of plasticity and assigns the effect of latency to the input impulse train. RMtrace along with the activity trace of input spike decide the amount of inhibitory complex released. This inhibitory complex decides the effective release of synaptic vesicles from the presynaptic region. Thus, inhibitory complex concentration along with the concentration of neurotransmitter release and the activity trace of input spike decide the magnitude associated with the released synaptic current. This value is the synaptic weight and its rate of change gives the synaptic efficacy, a parameter that describes the strength of plasticity.

The equilibrium point of inhibitory complex is adjusted because the value considered has a very high threshold and is impractical, to a value where the effects of the impulses can be observed distinctly. The state variables of the presynaptic region: effective strength of feedback of Retrograde Messenger (RMtrace), Inhibitory complex concentration (Inh) and activity trace of spike (C) and the concentration of neurotransmitter (D) are defined using the Tsodyks Markram Model as:

$$( Equation 1 ; subunit 28 in Fig . 2 )$$

$$\frac{d}{dt} RMtrace = - \frac{RMtrace}{{τ}_{r}} + RM$$

$$( Equation 2 ; subunit 30 in Fig . 2 )$$

$$\frac{d}{dt} Inh = - \frac{Inh}{{τ}_{inh}} + RMtraceC$$

$$( Equation 3 ; subunit 32 in Fig . 2 )$$

$$\frac{d}{dt} C = - \frac{1}{{τ}_{c}} [ C + Δδ ( t - {t}_{p} ) ]$$

$$( Equation 4 ; subunit 34 in Fig . 2 )$$

$$\frac{d}{dt} D = - \frac{1}{{τ}_{d}} [ D + \sum δ ( t - {t}_{d} ) ]$$

where, τᵣ is the time constant associated with the influx of RMS, τᵢₙₕ is the time constant associated with inhibition of release of neurotransmitters, tₚ is the time when an impulse is received at the presynaptic neuron, RM is the concentration of retrograde messenger present in the postsynaptic region and τ_{c} and τ_{d} are the time constants associated with the biological latency in activity spike and neurotransmitter release respectively. The first three equations give the resulting effect of the action potential on the concentration of the inhibitory complex. The fourth equation gives the concentration of neurotransmitter released from the presynaptic neuron which depends on the rate at which impulses enter. A is the parameter for Hebbian plasticity and is calculated according to Table 1.

TABLE 1
Specific Values of Timescales, Threshold Values
and Control Parameters in Neuron Model

| u[n] | sgn(RMtrace − RMrest) | Δ |
|---|---|---|
| 1 | 1 | 1 |
| 1 | 0 | −1 |
| 0 | 1 | −1 |
| 0 | 0 | 0 |

The probability of inhibition and release of neurotransmitter, concentration of neurotransmitter and synaptic current are written as:

$${P}_{inh} = {e}^{{e}^{- \frac{1. \times {10}^{- 7}}{inh}} - 1}$$

$$( Equation 6 ; subunit 36 in Fig . 2 )$$

$${P}_{rel} = {P}_{init} ( 1 - {P}_{inh} )$$

Here, the probability of inhibition is a function dependent only on the concentration of the inhibitory complex. The probabilities considered here decide the synaptic weight of a neuron i.e., the amount by which the transmission of an impulse through the synapse is magnified. The rate of change of synaptic weight is called synaptic efficacy and is calculated as:

$$( Equation 7 ; subunit 38 in Fig . 2 )$$

$$\frac{d}{dt} ω = {P}_{rel} . C . D$$

Hence, the resulting synaptic current transmitted can be written as:

$$I syn ( t ) = ω \sum \frac{t - {t}_{p}}{τ} {e}^{\frac{t - {t}_{p}}{τ}} δ ( t - {t}_{p} )$$

The postsynaptic region 16 can be a postsynaptic component that is digitally logically modelled. At the postsynaptic RM, the RM depends on the calcium concentration due to endoplasmic reticulum and secondary mediator. Calcium effects due to endoplasmic reticulum and secondary mediator can be written as:

$${τ}_{{C}_{c}} \frac{{dC}_{c}}{dt} = - {C}_{c} - {C}_{4} . f ( {C}_{c} , {C}_{c} ) + [ r + α ( {W}_{post} - {W}_{{post}_{rest}} ) + β {S}_{m} ] + k . \frac{d m}{dt}$$

$$τ {C}_{c} \text{?} {C}_{c} \frac{{dC}_{c}}{dt} = \text{?} ( {C}_{c} , {C}_{c} )$$

$$\text{?} ( {C}_{c} , {C}_{c} ) = {C}_{1} \frac{{C}_{c}^{2}}{1 + {C}_{c}^{2}} - \frac{{C}_{c}^{2}}{1 + {C}_{c}^{2}} . \frac{{C}_{c}^{4}}{{C}_{2}^{4} + {C}_{c}^{4}} - {C}_{3} {C}_{c}$$

$$\frac{d m}{dt} = {v}_{{1}_{max}} \text{?} . \frac{m}{1 + m}$$

$${τ}_{Sm} \frac{dSm}{dt} = ( 1 + tanh ( {S}_{Sm} ( {I}_{syn} - {I}_{{S}_{m}} ) ) ) ( 1 - Sm ) - \frac{Sm}{{d}_{Sm}}$$

$$\text{?} \text{indicates text missing or illegible when filed}$$

Here, in these equations, c1, c2, c3 and c4 are the fixed control parameters of the function f(cc,ce), cc describes the calcium concentration in the cytoplasm, ce represents the calcium concentration in the internal store (endoplasmic reticulum ER), Wpost is the recovery variable for the postsynaptic current in FHN model, Sm is the generation of IP3 in response to the influx of calcium current. The term

$$[ r + α ( Wpost - Wpostrest ) + β \cdot Sm ]$$

represents the calcium influx from the external space. Also, interaction between the cytoplasmic calcium (cc) and endoplasmic calcium (ce) is described with a two-variable function f(cc,ce). There is threshold value for the Sm production that is triggered by the synaptic current. Threshold parameter ISm is thus selected to distinguish between activated and inactivated states of the variable.

The plasticity of the system is decided by the retrograde messenger 14. The concentration of retrograde messenger 14 is only dependent on the postsynaptic calcium concentration. The concentration of postsynaptic retrograde messenger 14 is written as:

$$RM = {e}^{- e \sqrt{3} lo g ( {C}_{c} ) + 25.4146}$$

By FHN model, a simplified version of the Hodgkin-Huxley model, for postsynaptic site, is:

$$\begin{matrix}\frac{{dW}_{post}}{dt} = {V}_{post} + {I}_{post} - {I}_{syn} \\ \frac{{dV}_{post}}{dt} = {V}_{post} - \frac{{V}_{post}^{3}}{3} - {W}_{post}\end{matrix} \qquad ( Equation 15 ; subunit 38 in FIG . 2 )$$

The equations are optimized to improve the computational efficiency of the model and reduce its implementation cost by implementing polynomial expansion of functions to reduce complex and lengthy functions. This make the hardware logic easier to design and implement, with approximations are taken such that they resemble the curve closely in the domain the functions shall be operating in.

For the presynaptic region 12 (and component), because 1st order Tsodyks Markram differential equations are used, since all the equations are linear, there is no requirement to make any adjustments to them. However, for determining the probability of inhibition, we encounter the implementation of exponential function.

On using a CORDIC algorithm to implement the probability of inhibition, the area of the hardware is increased by a significant amount. Also, since the CORDIC algorithm operates on convergence, this would lead to an increase in time taken per process for computation. Hence, a solution to this problem is to use an rectangular hyperbolic function that closely resembles the curve in the region of operation.

$${P}_{inh} = \frac{- 0.00007}{Inh - 0.00001} + 1.1 \qquad ( Equation 16 ; subunit 40 in FIG . 2 )$$

For synaptic current, we consider the summation segment as a reset enabled function. Thus, we obtain a pair of equations Y and Z, which generate the form of unweighted synaptic current:

$$\begin{matrix}Y = \frac{t - {t}_{p}}{τ} {e}^{\frac{t - {t}_{p}}{τ}} \\ Z = τ {e}^{\frac{t - {t}_{p}}{τ}}\end{matrix}$$

For a pre-fixed frequency of tₚ₀ interval, the summation symbol in is removed without any loss of accuracy by considering the summation of each power for a finite number of terms. However, for SyNC model to function for any form of tₚ provided, we needed to make some approximations. Adoption of a piece-wise approach leads to a trade-off between area efficiency and error efficiency. Also, the properties of the curve are lost at every new impulse completely, which leads to significant error in output, unless more functions are added to compensate the loss. But this almost doubles the area. Hence, the alternative is to go for differential form of these equations. As such, the impulse driven activation can be enabled, without removing the value of Y completely before the activation. For this particular set of equations, activation conditions can be met simply by adding 1 to Z, when an impulse arrives in each cycle. Such a form is more robust in error handling for discretized calculations. Based on above technique, Isyn is calculated as follows:

$$\begin{matrix}\frac{dY}{dt} = \frac{Z - Y}{τ} \\ \frac{dZ}{dt} = \frac{- Z}{τ} + u [ n ]\end{matrix} \qquad ( Equation 18 ; subunit 42 in FIG . 2 )$$

$$I syn = ω \cdot Y \qquad ( Equation 19 ; subunit 44 in FIG . 2 )$$

For the postsynaptic region 16 (and component), the value of f(C_{c}, Cₑ) is in the order of 10-12, hence making the detection of this potential via instruments impossible. Moreover, the value of the function is much lower than the noise 442 threshold, due to which the noise parameter will be the primary contributor to the value of this function. Hence, we consider f(c, E)=0. This in turn gives us E=0. Hence, the changed calcium dynamics equations are:

$${τ}_{{C}_{c}} = \frac{{dC}_{c}}{dt} = - {C}_{c} + [ r + α ( {W}_{post} - {W}_{{post}_{rest}} ) + β {S}_{m} ] + k \cdot \frac{d m}{dt} \qquad ( Equation 20 ; subunit 46 in FIG . 2 )$$

$$\frac{d m}{dt} = {v}_{{1}_{ma x}} {C}_{c}^{2} {K}_{d}^{2} ( 1 - \frac{{C}_{c}^{2}}{{K}_{d}^{2}} + \frac{{C}_{c}^{4}}{{K}_{d}^{4}} ) - {v}_{{2}_{ma x}} \cdot k 1 \cdot ( 1 - m + {m}^{2} ) \qquad ( Equation 21 ; subunit 48 in FIG . 2 )$$

$$k 1 = \frac{{[ {Na}^{2 +} ]}^{2}}{{K}_{{Na}^{2 +}}^{2} + {[ {Na}^{2 +} ]}^{2}}$$

For secondary mediator, polynomial expansion of tan h is implemented to reduce the complexity of the equation:

$${τ}_{Sm} \frac{dSm}{dt} = ( 1 + {k}_{s} - \frac{{k}_{s}}{3} ) ( 1 - Sm ) - \frac{Sm}{{d}_{Sm}} \qquad ( Equation 22 ; subunit 50 in FIG . 2 )$$

$${k}_{s} = {S}_{Sm} ( {I}_{syn} - {I}_{Sm} )$$

For concentration of retrograde messenger, we implement polynomial expansion of exponential function:

$$RM = 1 - ( k 2 \cdot {c}^{k 1} ) + \frac{{( k 2 \cdot {c}^{k 1} )}^{2}}{2} - \frac{{( k 2 \cdot {c}^{k 1} )}^{3}}{6} \qquad ( Equation 23 ; subunit 52 in FIG . 2 )$$

where k1=2.0447, k2=9.1799×10⁻¹².

The original FHN model can then be modified such that the nonlinear terms are eliminated. Unlike the other equations where powers of a variable in expansion are greatly problematic, this scenario is avoided here since one can reduce power taken in this particular variable description with no noticeable precision loss. This enables one to approximately solve both linearized FHN equations for the power cost of one. So, the nonlinear term from:

$$f ( {V}_{post} ) = {V}_{post} - \frac{{V}_{post}^{3}}{3}$$

can be rewritten into the closest fitting linear curve. An exemplary curve here is:

$$f ( {V}_{post} ) = 1 - \frac{\vert {V}_{post} \vert}{2} \qquad ( Equation 25 ; subunit 38 in FIG . 2 )$$

FIG. 2 is a diagram of one embodiment of a hardware circuit implementing digital logic for at least partial instantiation of the synaptic model, with a description of data flow in at least partially a hardware architecture. Each submodule for its corresponding equation is highlighted. The architecture has the lowest power requirements for the complexity of the designed synaptic model.

Large scale hardware usage is key for further understanding the impact of plasticity and synaptopathy mechanisms on larger circuits. Use of the IEEE 754 single precision floating point has accuracy within tolerable error ranges. It is also remarkably small and efficient in terms of PPA (power, performance, area) versus ARM core implementations. It has been a prevalent practice of late to use floating point numerals in 16-bit widths or lower order custom width floats to optimize computational resources specifically for edge computing use-cases. Such practices give us higher power efficiency which increases the scalability of the neuronal circuit model. However, these extant ARM architectures were found to provide a decisive loss in accuracy within the neuron ASDint model. Due to such considerations, IEEE-754 single precision floating point is the more optimal suitable bit width. For comparison of potential neural network modeling, one of the well-known state-of-the-art SNN architectures, IBM TrueNorth, and Intel Loihi has been considered.

IBM TrueNorth uses a smaller technology node of 28 nm and consumes about 100 mW of power. It has a power density of 20 mW/cm2 consisting of 5.4 billion transistors. Intel Loihi has used a further smaller node of 14 nm processor with a 2 billion transistor size or 60 mm² in chip area. The proposed SyNC architecture is designed on a technology node of 45 nm and consists of 9.4 million transistors over an area of 1.877 mm². Hence, here, one can bypass the requirements of programmability to model diverse descriptive synaptic models by design of a minimalist ASIC synaptic core that describes every state variable in a synapse. On utilization of lower technology nodes, performance of the system is at least at par with state-of-the-art SNN architectures. The present system can also be implements on other programmable Application-Specific-Processors for better flexibility and programmability and to have a one chip for all solutions.

Computer models built in equivalent Posit numeral representation have demonstrated highly flexible and fast convergence in specific format scientific functions. Such models have shown accuracy comparable to IEEE-754 double precision floating point, using much lesser resources than the latter. However, due to complications in evaluations that result from dynamic power owing to higher switching of states, the net power requirement is significantly high on account of deep pipeline and conversion of FPGA fabric-optimized design to general ASIC.

Here, the computation core has been developed to work with IEEE-754 single precision floating point number system. The pathways of data flow in the hardware realization is further described in FIG. 2.

The hardware design has been pipelined with basic pipeline registers and clock speed has been achieved as high as 1 GHz in 45 nm technology node. The hardware design of the of this model, with exact same input configurations, have been simulated in Questasim 10.0b Simulator by simulation scripts and output results are functionally verified by self-developed utility scripts in C++ v.11 and Python 3.8. For simulation, as well as the hardware design for the given system of equations, a constant input of retrograde messenger 14 from the presynaptic region 12 such that it is always just above the threshold for activation of synaptic plasticity parameter. The synaptic model was implemented at register-transfer-level (RTL) which is mapped and synthesized using Synopsys Design Compiler on 4 nm OpenNangate technology. The power consumption is derived using a Synopsys Power Compiler. Standard operating frequencies of almost 1 GHz is met for all the designs in 45 nm ASIC.

For low power, economical and optimized implementation of most widely used single precision float, one can use reduced complexity hardware of base arithmetic units. Complexity of other numerous subunits are also significantly reduced. This brought down the power and area footprints by orders of magnitude than the base version to 2.135 W and 1.877 mm² respectively, by reduction of the number of intermediate registers and less frequent switching of intermediate variables. Through extensive pipelining, single precision floating point optimized version can meet an acceptable operating frequency of 1030 MHz.

One of the key features of the present mathematical design is that an endocannabinoid feedback mechanism is involved. While this is a staple in analog designs, for hardware implementation, an inadvertent delay is obtained in the system from inclusion of the endocannabinoid feedback mechanism data. This is normally observed to be equivalent to 25 clock cycles in the hardware domain. Here, instead of resorting to Verilog AMS, the accuracy of the model to control and minimize this delay to negligible extent by varying δ. The role of δ in our model is defining the relationship between the clock timing and the actual timeframe of operation. Hence by increasing δ, we decrease the effective influence of the delay in the timeframe of operation of the synapse.

A significant advantage of the at least partial hardware implementation of the logic in the present system is use in large scale hardware implementations. Such applications necessitate low power consumption designs. In the past, solutions such as linearization have been applied to obtain the required level of biological complexity but with reduced mathematical complexity. In the variable outputs obtained from the hardware realization of the present neuron model, a low latency in the results is observed. The net latency across all variables is 0.031 s, which is an effect of pipelining as well as the delay in feedback of retrograde messenger 14 to the presynaptic region.

As can be observed from the equations, while other equations have some arbitrary and unusable value output before the system stabilizes for accepting action potential input, the only variable that does not always return to rest and which impacts the variables that come after it is synaptic weight, w. For this very reason, the first values obtained just during the initialization of the model within this period must be rejected and the value of w is fixed at 0, to remove the effect of default values of w from influencing the nature of the curve. The error is less than 1% for architectures designed, which are sufficiently low and the output is barely affected and hence acceptable.

The performance of this hardware design with precision mode IEEE-754 Single Precision floating point, the amplitudes of the variables are higher in the hardware realization than that of pure software calculated results. This is a result of using differential forms of the equations to develop the hardware realization. The response of the variables in the current curves is a consequence of the choice of value of δ for model implementation. The smoothness and root mean square (RMS) accuracy of all obtained curves are directly proportional to δ. Hence, the hardware model is designed with such considerations.

However, for a biological synaptic system, the currency of communication is not entirely the form of the curve, but also the concentration of calcium influx associated with it. Rather, after RMS error reaches a particular range, it is this concentration influx which is more important to replicate. This can be evaluated by obtaining the error in area under the curve in the form of Area Average Error (AAE). In the present model, the conservation of molecules involved is of higher importance from a biological perspective, therefore one should disregard the amplitude error in favor of lower AAE after RMS error is within 1 percent.

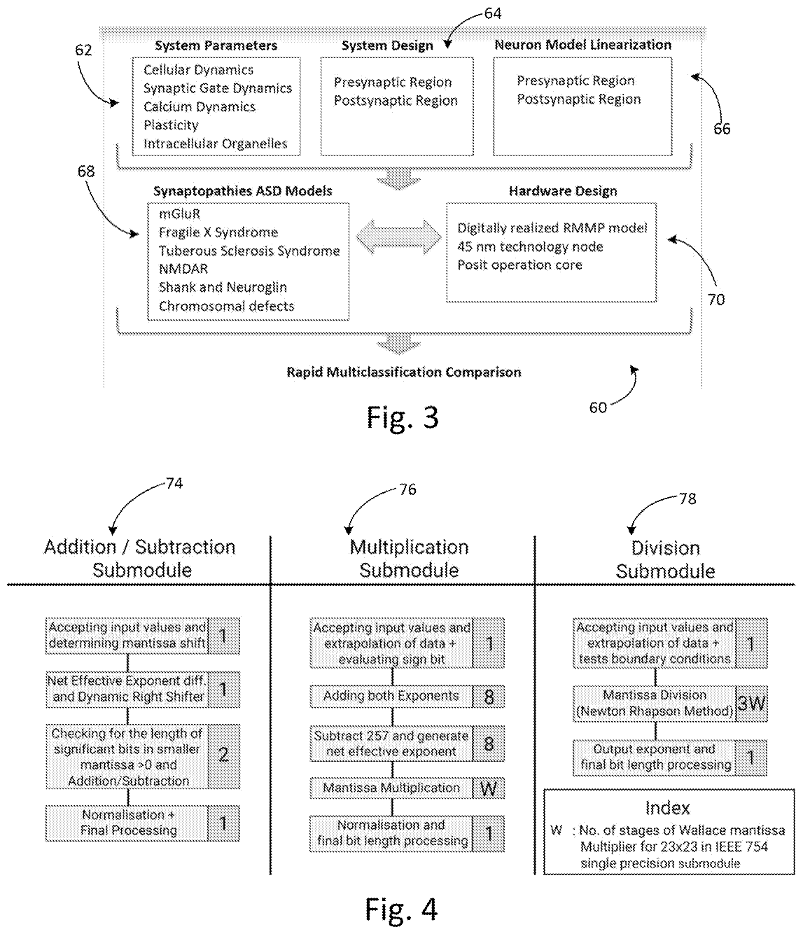

FIG. 3 is an object diagram 60 illustrating the objects comprising one embodiment of the software/hardware architecture of the present invention. The hardware is preferably used with neural algorithms to relay ASD complexity. The objects, which can be modeled in software, hardware, or preferably a combination thereof include a systems paraments module 62, that incudes, at least, in first layer attributes for cellular dynamics, synaptic gate dynamics, calcium dynamics, plasticity, and intercellular organelles. There is also a system design module 64, which includes the presynaptic region 12 (which can be a presynaptic component) and postsynaptic region 16 (which can be a postsynaptic component). A neuron model linearization submodule 66 also contains the presynaptic region 12 and postsynaptic region 16 attributes for incorporation in the model.

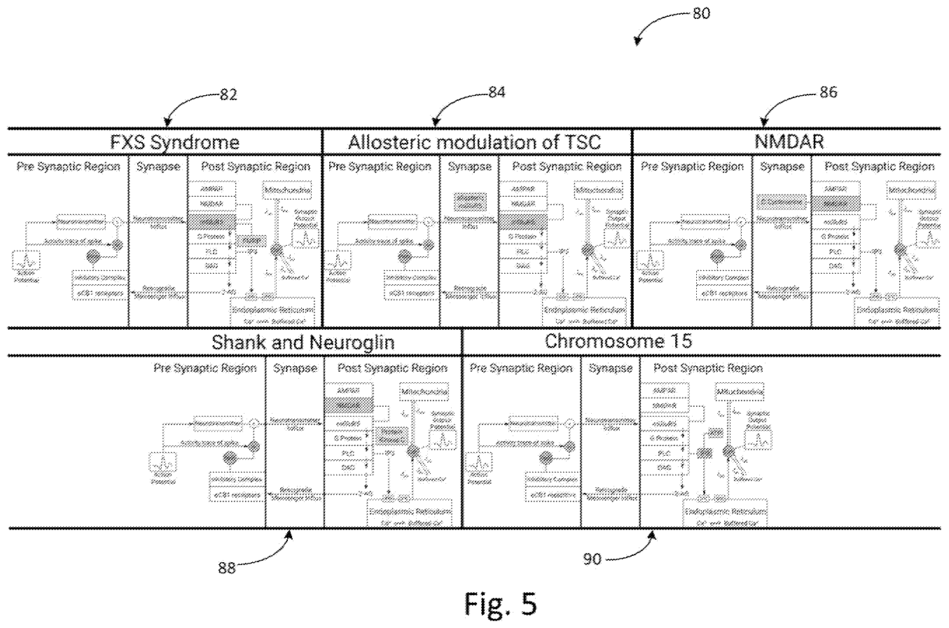

The objects of the second layer of the model include a synaptopathies ASD models module 68, which includes the various models described with respect to FIG. 5. In the second layer is also a hardware design module 70 that include the hardware attributes such as the digitally realize RMMP model, the logic hardware nodes, and a posit operation core. These objects can all be instantiated at once or partially, thus utilizing one, several, or all of the ASD models in predicting or analyzing synaptic functionality, predictive pharmacological effect, or synaptopathies.

FIG. 4 is a diagram of three arithmetic submodules used in the present software architecture, with each task representing the number of pipelined stages involved for that task in the hardware architecture. FIG. 4 describes the different stages of pipelining in terms of registers or flip-flops in each arithmetic unit used in the model. The design can be pipelined extensively and even arithmetic operators have been pipelined for mantissa operations by using custom-made, non-compressed, tree structured, pipelined, moderately large sized and power consuming high speed, Wallace-tree multipliers. The largest combinational unit floating point multiplier architecture is a mantissa multiplication of respective mantissa widths, whereas the largest one in division is the iterative Newton-Raphson division method which consists of three mantissa width multiplications for both posit and float. All of these multiplications are done using custom implemented wallace tree unsigned multipliers for gaining greater speed and to meet acceptable operating frequency.

Thus, in FIG. 4, there is an addition/subtraction submodule 74 that effects the mantissa shift of exponents for addition and subtraction, and a multiplication submodule 76 that effects one embodiment of mantissa-modified multiplication. A separate division submodule 78 which includes a Newton Rhapsod method for mantissa division.

FIG. 5 is a representational diagram 80 of the representation of affected components and modulators in the model for each considered Autism Spectrum Disorder (ASD) pathway scenario. ASDs form a group of diverse neurodevelopmental conditions defined by two core symptoms: social deficits that include communication and interaction impairments; and stereotypical, repetitive, and restricted behaviors. One of the main features of ASD is the high level of heterogeneity resulting in complexity both when considering symptoms and causative factors. It is possible that every single disorder within the ASD group has its unique mechanisms and consequences, with environmental and genetic factors playing roles in the etiology of ASD. In that regard, computational models help compare and categorize ASD effects on synaptic function.

The present system and method therefore allow the modeling of several multiple ASD mechanisms. It is in fact possible that every single disorder within the group has its unique mechanisms and consequences, with environmental and genetic factors playing roles in the etiology of ASD. As shown in FIG. 5, the present system and multiple ASD mechanisms in mathematical descriptions that incorporate various regions of the synapse.

In one example, the present system can model the potential synaptopathology of Fragile X Syndrome (FXS) (Module 82 in FIG. 5). In the model, consideration is given to the role of Fragile X protein, FMRP, in mRNA translation regulation and binding, which is decisive in determining the quality of synaptic communication between the presynaptic and postsynaptic regions. Alterations in signaling, expression, and function of group I mGluRs are related to neurodevelopmental disorders). Group I mGluRs comprising mGluRI and mGluR5 are at least partial regulators of syndromic and non-syndromic forms of ASD, making them potential therapeutic targets. There are two distinct groups of mGluRs. Some groups of mGluR couple with Gq proteins and are involved in excitatory neural communication, like mGluR1 and mGluR5. The rest of the mGluR groups are involved in non-excitatory neural communication without assistance from Gq proteins. mGluR5 signaling was shown to be affected in opposing directions in both Fragile X Syndrome (FXS) and Tuberous Sclerosis Complex (TSC). The response of these variations onto secondary mediator dynamics in the model are directly related to mGluR5 dynamics. Activation of group 1 mGluR mediates the release of Ca2+ in the postsynaptic region through the activation of kinases such as mTOR and ERK. These kinases are involved in the modulation of Homer 1, a protein generation in the nucleus, which in turn modulates the mGluR53 receptor dynamics through its companion protein Homer 1 b/c. FMRP in this mechanism plays the role of a inhibitor responsible for attenuating the production of Homer 1a. However, in the absence of FMRP, there is an abnormal increase in mGluR5 receptor dynamics. The consequent excessive mGluR-LTD constitutes the aberrations observed in FXS.

FXS is the most common monogenic form of inherited intellectual disability. In a majority of the cases observed, the cause of hampered FMRP dynamics is the expansion of the CGG trinucleotide, repeating in the five untranslated region of the Fragile X FMRI gene. When the repetition of CGG is as high as more than 1,000 times, this segment of the FMRI gene undergoes methylation, effectively silencing gene activity and consequently, the generation of FMRP protein. FMRP suppresses mGluR5 receptor dynamics in the postsynaptic region and absence of FMRP leads to exaggerated synthesis of proteins required for mGluR-dependent LTP, thereby enhancing its magnitude.

For modelling the effect of FMRP in FXS syndrome in a synaptic device, one can consider its impact on mGluR5 LTD. The equilibrium dynamics for the mGluR5 are defined due to accelerated generation of Homer 1a. The synaptic current influx in the postsynaptic region can be considered a directly proportional to mGluR5 activity. Since the increased sensitivity in dynamics is observed, the gain achieved can be expressed as:

$$\frac{dInflux}{dt} = - {k}_{fmrp} \cdot {k}_{t} \cdot [ {I}_{syn} ]$$

$${I}_{syn}^{′} = {I}_{syn} + Influx$$

Here, the dynamic parameter K_{fmrp} is the outcome of molecular stochastic process. Therefore, it behaves as the source of noisy behavior in this model for FMRP concentration. The variations in FXS are expressed first in secondary mediator in the model.

Another inherited intellectual disability associated with ASD is Tuberous Sclerosis Syndrome (TSC) (Module 84 in FIG. 5). The syndrome is caused by heterozygous mutations in genes of the TSC1/2 complex involved in mTOR mediated signaling that couples cell surface receptors to protein synthesis. Several studies of mouse models of TSC in mouse models have exhibited reduction in decreased mGluR5-mediated LTD caused by impaired protein synthesis. Consistently, treatment with an allosteric mGluR5 agonist was able to restore mGluR5-mediated LTD. Both positive and negative allosteric modulation of mGluR5 have been proposed as a therapeutic intervention in neurodevelopmental disorders. Negative allosteric modulators (NAMs) of mGluR5 have been shown to alleviate long-term memory deficits, excessive repetitive behaviors, motor stereotypes and social interaction abnormalities in various models of autism. The mGluR5 positive allosteric modulator (PAM) was reported to ameliorate deficits in learning and memory and chemically induced hippocampal LTD in Tsc2+/mice.

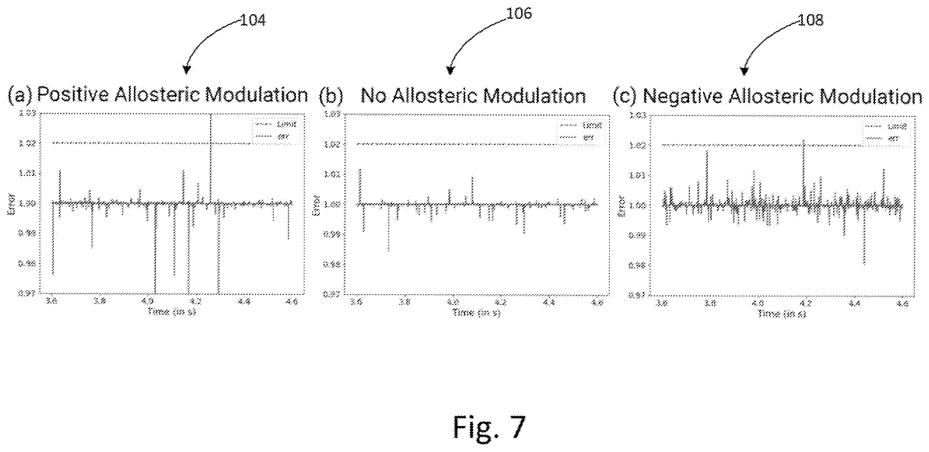

To model TSC, allosteric modulation is preferred. Due to lack of an exact model of how both enhancement and inhibition of mGluR5 activity improves synaptic output in allosteric modulation of TSC (further shown in FIG. 7), a mathematical form can duplicate such behavior successfully. Hence, in the model, the behavior of the NAM is akin to lowering the threshold of mGluR5 activation, leading to easier activation of the postsynaptic region and improving mGluR-LTP. The equivalent mechanism of the action of PAM is considered in the impact of impaired protein function behavior onto the output dynamics of the synapse. As observed from numerous studies, the activity of TSC is essentially centered around the activity of mGluR5. This is consistent with mGluR5 PAM alleviation of LTP by compensating for reduced mGluR5. However, mGluR5 must also be the primary carrier of distortion in this system because reduction of mGluR5 activity by NAM is also beneficial for the plasticity of the system. Hence, the impaired protein synthesis is the generator of distortion in the process. The equilibrium constructs of the system are:

$$\frac{{dk}_{mGluR 5}}{dt} = + {k}_{mGluR5} - {k}_{namGluR5}$$

$$\frac{{dk}_{mGluR 5}}{dt} = + {k}_{mGluR5} + {k}_{pamGluR5}$$

$${k}_{namGluR5} > {k}_{mGluR5} > {k}_{pamGluR5}$$

Another synaptopathological model is NMDAR and the partial agonist 0-cycloserine (Module 86 in FIG. 5). The NMDAR is a glutamate channel protein and ion receptor at the synapse. The implication of NMDARs in the etiology of ASD has been supported by both clinical and non-clinical studies. Clinical studies have identified genetic variants in the GRIN2A and GRIN2B genes encoding the GluN2A and GluN2B subunits of the NMDAR, respectively. It is highly plausible that differences in subunit composition affect functional properties of NMDARs and/or NMDAR-dependent plasticity. A role for NMDARs in ASD is supported by the fact that social withdrawal and repetitive behavior in individuals with ASD can be alleviated by the NMDAR co-agonist D-cycloserine. By contrast, NMDAR antagonists memantine and amantadine improve ASD-related symptoms. Thus, ASD could result, at least in part, from deviations in hormetic NMDAR responses

D-cycloserine is considered to be a partial agonist of NMDAR i.e., it acts like an agonist when it is the primary neurotransmitter involved but has antagonistic features when it is abundant in the system. This seems to be due to its different receptor subtype selectivity and intrinsic action, which depends on various NR2 subunits (NR2A, NR2B, NR2C), which happen to be the location of glutamate binding. One of the most prevalent hypotheses suggested is that the effects seen in vivo at low doses of D-cycloserine reflect its agonistic action at the NR1/NR2C receptors, for which it has a high affinity, while at high doses the effects might be due to antagonistic inhibition of NR1/NR2A and NR1/NR2B receptors, for which D-cycloserine has a lower affinity. It is observed that this effects the glutamate binding and hence does not affect the plasticity from its own path. The resultant glutamate binding is inhibited at NR2A, NR2B once all sites of NR2C are occupied by either of the agonist. It must be noted that D-cycloserine in natural state is not an activated pathway, hence making it the noise source in the system.

For modeling NMDAR with a partial agonist, D-cycloserine's activity as a partial agonist can be best described as a competitive process at NMDAR. Electrodynamically, in the concerned scenarios, the concentration of D-cycloserine is more than the concentration of NMDAR, hence the molecules unable to bind with NR1/NR2C receptors act as agonists. However, it is also observed that this effects the glutamate binding and hence does not affect the plasticity from its own path. The resultant glutamate binding is inhibited at NR2A, NR2B once all sites of NR2C are occupied by either of the agonist. It must be noted that D-cycloserine in natural state is not an action potential activated pathway involved in synaptic transmission dynamics, hence making it the noise source in the system. To demonstrate the role of D-cycloserine, its role in synaptic transmission dynamics via the competitive concentration dynamics process is modeled. The effect of such dynamics can be first observed at the secondary mediator, the first state variable evaluated at the postsynaptic region and is directly dependent to mGluR5 activity. To better understand the process dynamics, the distortion for this specific case is evaluated by comparison between the simulated outputs to a noisy signal for the case where D-cycloserine is acting passively along with primary neurotransmitter (glutamate). For the scenario involving D-cycloserine along with glutamate:

$$\frac{dIsyn}{dt} = [ + \frac{Isyn}{{k}_{Glutamate}} - \frac{N}{{k}_{D - cycloserine}} + \frac{1}{{k}_{adjust}} ]$$

Another synaptopathological model is called “Shank and Neuroglin” (Module 88 in FIG. 5). Shank proteins are master scaffold proteins within the postsynaptic density of glutamatergic neurons important for synaptogenesis and function, but have also recently been described to have a presynaptic role in Drosophila. Mutations in human Shank genes are also found in ASD and expected to relate to their role in activity-dependent formation and remodeling of synaptic function

Analysis in a mouse model of Shank2, lacking exons 6 and 7, showed reduced hippocampal NMDAR function, whereas mice lacking only exon 7 show up-regulation of NMDARs in synaptosomes, increased NMDAR/AMPAR ratio, and enhanced NMDAR dependent LTP. One can model this condition by varying the equilibrium constant value of k or kNMDAR. Hence, the synaptic weight limit is increased as the limit of excitation current is increased. Due to higher NMDAR/AMPAR value, an assumption is made that synaptic vesicle release is unbounded. However, two Shank2 deletion mouse models resulted in very similar social deficits, which support the notion that deviation in NMDAR function in either direction can result in ASD like phenotypes. Interestingly, aberrant NMDAR function and behavioral deficits observed in those mice could be normalized with systemic D-cycloserine and administration of the positive modulator of mGluR5 3-Cyano-N-1, 3-diphenyl-IH-pyrazol-5-ylbenzamide (CDPPB).

Shank1 similarly has been associated with ASD-like behavior in mice, including increased anxiety and deficits in contextual fear learning, but with improvements in spatial learning. This corresponds well with the cognitive changes observed in many individuals with ASD. Shank1 mice additionally show smaller dendritic spines in CA1 pyramidal hippocampal neurons and weaker synaptic transmission, supporting recent evidence that dendritic spine abnormalities are associated with ASD.

One can also consider Neuroglins, which are synaptic cell adhesion molecules in which several mutations have been found associated with ASD. Knocking-in the ASD-associated R451C substitution into the endogenous Neuroglins NLGN3 locus caused a prominent decrease in Neuroglin levels that resulted in impaired social behaviors, enhanced spatial learning, and increased synaptic inhibition in the mouse somatosensory cortex. Its effects are complementary to Shank, as in the ratio of AMPAR/NMDAR becomes lower. Shank and Neuroglin mechanisms can be modeled on the effective change in synaptic current and the impacted changes can be observed in the secondary mediator dynamics.

The role of Shank proteins in synaptogenesis and function, while understood to a certain extent, have not been successfully generalized in a model due to the numerous ways Shank proteins can impact the synaptic function. Many experimental studies have been made to understand its role, but the highly diverse and often contentious conclusions from these studies have been an impediment. Here, one can reduce the core properties of the Shank such that all such scenarios can be simulated successfully. The core properties of Shank protein can be reduced to

$$\frac{{Isyn}^{′}}{Isyn} = \frac{{k}_{Shank}}{. {k}_{Neuroglin}} \cdot {k}_{adjust}$$

variations in NMDAR activity, NMDAR mediated LTP and NMDAR/AMPAR ratio at the postsynaptic region. One can model this condition by varying the equilibrium constant value of k or KNMDAR. To be able to model a wide range of NMDAR/AMPAR values, the model adopts a working assumption that synaptic vesicle release is unbounded. The action of Neuroglins, synaptic cell adhesion molecules, are considered which have effects complementary to that of Shank, as in the ratio of AMPAR/NMDAR becomes lower. A composite model is developed that can effectively demonstrate the impact of Shank and Neuroglin dynamics on the effective change in synaptic current and the impacted changes can be observed in the secondary mediator dynamics:

A further potential synaptopathological model is for chromosomal defects, such as Chromosome 15 syndrome (Module 90 in FIG. 5). A “chromosome 15 phenotype” characterized by ataxia, language delay, intellectual disability, repetitive movement disorders and facial dysmorphic features has been described in individuals with chromosome 15 duplications. Within the 15q11-15q13 locus. One can model the chromosome 15 inhibitory receptor action on neuronal dynamics onto the synaptic mediator dynamics and observe their effect on the calcium dynamics in the postsynaptic region of the neuron as a consequence of ITP3K enzyme and ATP on IP3 dynamics.

A “chromosome 15 phenotype” is characterized by ataxia, language delay, epilepsy, intellectual disability, repetitive movement disorders and facial dysmorphic features and has been described in individuals with chromosome 15 duplications. To model this synaptopathy, the chromosome 15 inhibitory receptor action on neuronal dynamics onto the synaptic mediator is observed, specifically with the effects on calcium dynamics in the postsynaptic region of the neuron.

In general, synaptopathies can be described in the form of distortions observed in the system compared to normal conditions. The distortion obtained from the ASDint simulations show consistency with the conditions underlying those specific synaptopathies. Two primary ways to look at a noise response is the gain in potential strength they provide and the number of spikes in the resultant output that are at a threshold to impact the system dynamics. Gain in potential results in stronger and more sensitive LTP dynamics, reducing the threshold of action potential spike input and frequency required to activate it. Such an output response implies improved LTP dynamics and lower LTD threshold, resulting in greater sensitivity of the model as well as lower duration of information retention of the synapse. Greater noise spikes result in false activation of the region and can result in faulty activation of the boutons. Such impulses can result in variations in synaptic weight and lead to repetitive behaviors. In analysis of the ASD mechanisms different resulting distortions occurred. Evaluation of distortions provides insights into phenotypes observed in ASD synaptopathies. For TSC under the impact of negative allosteric modulation, the noise spikes in the distorted curve, although of lower amplitude threshold, are greater than the general scenario. This can manifest itself in the form of false activation of the postsynaptic region, which can be considered a trigger for repetitive behaviors.

When positive allosteric modulation occurs, one observes the distortion curve to have higher noisy spikes as well as lower attenuation of action potential. Hence, it has better LTP characteristics and LTD threshold that can be associated with obstructions in learning and lower attention span. Modelling the FXS syndrome in ASDint module, LTP is very easily activated here and can be observed in the form of lower LTP threshold. Thus, it can be associated with difficulty in learning process as well as increased sensitivity to noisy inputs in the neural circuitry. The interactions of partial agonist with NMDAR show us the output of competition in the synaptic channel manifested at the synaptic gate. Social withdrawal and repetitive behavior are associated with this particular synaptopathy. On observing the noise response of the ASDint module, the noise-induced variations are the highest when compared to the other three scenarios and can be considered a trigger for repetitive behaviour. For Shank, NG activity variations, the NMDAR induced LTP is affected and results in higher required amplitude of LTD, which we can see from the noise plots resembles the expected activity variations. Chromosome 15 syndrome is associated with repetitive movements which as observed from the distortion curve, could be a consequence of false activation of postsynaptic regions.

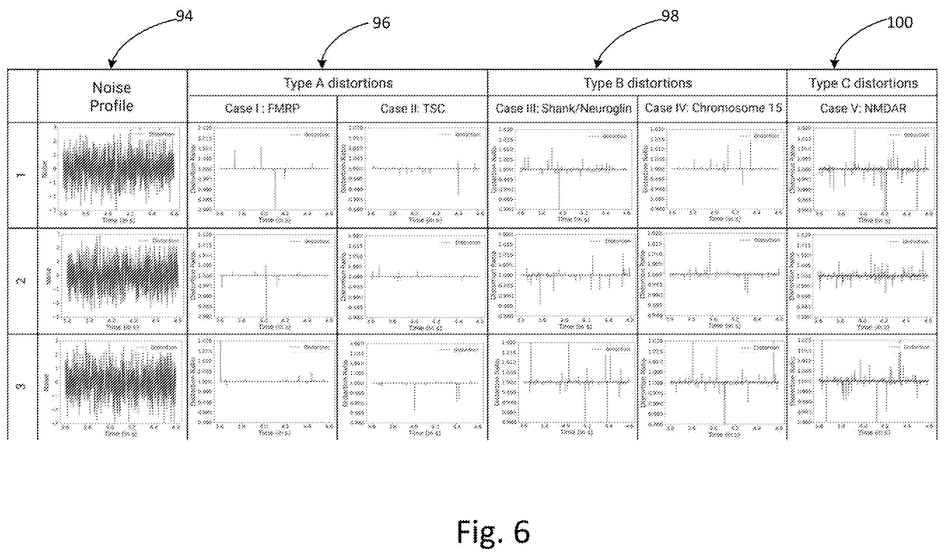

FIG. 6 is a classification diagram of ASD mechanisms considered based on distortion from the ideal synaptic output potential, with ideal distortion response set at 1. Noise profiles 94 describe the variations in variables that are considered for these synaptopathies, with baseline for no noise input set at zero. The baseline for noise is added to the plots for reference. D-cycloserine here acts as partial agonist on NMDAR. Type A synaptopathies, such as FMRP and TSC models, are categorized as the least amount of distortions (Type A distortions 96). Multiple neurons must be affected by these particular synaptopathies to be observable. The impact on one synapse can be considered unobservable by experimental methods. Hence, to have observable impact on the neuronal circuit, multiple synapses and neural networks with them must be affected by these particular synaptopathies.

Type B synaptopathies, such as Shank-Neuroglin and Chromosome 15 models, have observable impact on the neuronal circuit even when occurring in a small collective of such affected neurons (Type B distortions 98). Type B events show considerable distortions whose impact can be considered observable via high precision experimentations. To have observable impact on the neuronal circuit, a small collective of such affected synapses and networks are sufficient.

Type C synaptopathies, such as an NMDAR model, show abruptly high distortions that can lead to easily observable variations in the neuronal circuit (Type C distortions 100). Type C shows abruptly high distortions whose impact on a single synapse can be considered observable by experimental methods. Few such altered synapses for neurons in key processing regions of the brain will lead to observable variations in the neuronal circuit.

FIG. 7 is a diagram of graphs demonstration the effect of allosteric modulation in tuberous sclerosis complex (TSC). The effect of allosteric modulation in TSC. The limitations for positive allosteric modulation 104 and negative allosteric modulation 108 are compared with an unperturbed scenario in which no allosteric modulation 106 agents were introduced into the system. Positive allosteric modulation 104 results in higher amplitude and low frequency tfilter. Negative allosteric modulation 108 results in lower amplitude threshold and high frequency filter.

If purely implemented in software, the functions described may be stored on or transmitted over as one or more instructions or code on a non-transitory computer-readable medium and executed by a hardware-based processing unit. Computer-readable media may comprise computer-readable storage media (which corresponds to tangible media, such as data storage media) or communication media including, for example, any medium that facilitates transfer of a computer program from one place to another. In this manner, the computer-readable medium may generally correspond to (1) a non-transitory tangible computer-readable storage medium, or (2) a communication medium, such as a signal or carrier wave. A data storage medium may be any available medium that can be accessed by one or more computers or one or more processors to retrieve instructions, code and/or data structures for implementing the techniques described in this disclosure. The computer program product may include a computer-readable medium.

By way of example, and not limitation, such computer-readable storage media can comprise RAM, ROM, EEPROM, CD-ROM or other optical disk storage, magnetic disk storage or other magnetic storage devices, flash memory, or any other medium that can be used to store desired program code in the form of instructions or data structures and that can be accessed by a computer. Also, any connection is properly termed a computer-readable medium. For example, if the instructions are transmitted from a website, server, or other remote source using a coaxial cable, fiber optic cable, twisted pair, Digital Subscriber Line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes Compact Disc (CD), laser disc, optical disc, Digital Versatile Disc (DVD), floppy disk and blu-ray disc where disks usually reproduce data magnetically, while discs reproduce data optically with lasers. Combinations of the above should also be included within the scope of computer-readable media.

The instructions held in the software may be executed by one or more processors, such as: one or more Digital Signal Processors (DSPs), general purpose microprocessors, Application Specific Integrated Circuits (ASICs), field programmable logic arrays (FPGAs), or other equivalent integrated or discrete logic circuitry. Thus, the term “processor,” as used herein may refer to any of the foregoing structure or any other structure suitable for implementation of the techniques described herein. Additionally, in some aspects, the functionality described herein may be provided within dedicated hardware and/or software modules configured for encoding and decoding, or incorporated in a combined codec. Furthermore, the techniques may be fully implemented in one or more hardware circuits or logic elements.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to, possessing only those one or more steps or elements. Likewise, a step of a method or an element of a device that “comprises”, “has”, “includes” or “contains” one or more features possesses those one or more features, but is not limited to, possessing only those one or more features. Furthermore, a device or structure that is configured in a certain way is configured in at least that way but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A system for modeling neuronal synaptic functionality, comprising:
a computation core comprised of one or more high-speed processors; and
a synaptic model configured by, at least:
a presynaptic component including a presynaptic target having, at least, a plasticity parameter and activity spike strength;
a retrograde signaling component including a retrograde messenger that selectively generates a molecular uptake signal;
a postsynaptic receptor component; and
wherein the retrograde messenger acts on a presynaptic target to modulate the plasticity parameter and activity spike strength of the presynaptic component based upon a calculated molecular uptake at the postsynaptic receptor component, the retrograde messenger thereby generating the molecular uptake signal and transmitting the molecular uptake signal to the presynaptic component.

2. The system of claim 1, wherein the molecular uptake signal of the retrograde signaling component is modulatable in both duration and latency, and the retrograde signaling component further determining an uptake and degradation of molecules at the postsynaptic receptor component and modulating the molecular uptake signal based upon such determination.

3. The system of claim 1, wherein the retrograde messenger is located at the postsynaptic component and losslessly transmits the molecular uptake signal to the presynaptic component.

4. The system of claim 1, wherein the postsynaptic receptor components includes, at least, models for ionotropic glutamate receptors (iGluR) and metabotropic glutamate receptors (mGluR).

5. The system of claim 1, wherein the synaptic model is for calcium-mediated synaptic events.

6. The system of claim 1, wherein the synaptic model further models synaptic strength as influenced by presynaptic and postsynaptic activity in an activity-dependent synaptic plasticity process.

7. The system of claim 1, wherein the synaptic model is a neural net.

8. The system of claim 7, wherein the computation core further includes a digital logic that at least partially instantiates the synaptic model.

9. A method of modeling neuronal synaptic functionality, comprising:
configuring a synaptic model on a computation core comprised of one or more high-speed processors, by at least the steps of:
configuring a presynaptic component including at least a presynaptic target having, at least, a plasticity parameter and activity spike strength;
configuring a retrograde signaling component including a retrograde messenger;
selectively generating a molecular uptake signal from the retrograde messenger;
configuring a postsynaptic receptor component;
modeling a presynaptic target at the retrograde messenger by modulating the plasticity parameter and activity spike strength of the presynaptic component based upon a calculated molecular uptake at the postsynaptic receptor component; and
transmitting the molecular uptake signal to the presynaptic component.

10. The method of claim 9, wherein the molecular uptake signal of the retrograde signaling component is modulatable in both duration and latency, and further:
determining, at the retrograde signaling component, an uptake and degradation of molecules at the postsynaptic receptor component; and
modulating the molecular uptake signal based upon such determination.

11. The method of claim 9, wherein the retrograde messenger is located at the postsynaptic component, further losslessly transmitting the molecular uptake signal to the presynaptic component.

12. The method of claim 9, further comprising modeling ionotropic glutamate receptors (iGluR) and metabotropic glutamate receptors (mGluR) at the postsynaptic receptor components.

13. The method of claim 9, wherein configuring a synaptic model is further configuring a synaptic model for calcium-mediated synaptic events.

14. The method of claim 9, wherein configuring a synaptic model is further configuring a synaptic model for synaptic strength as influenced by presynaptic and postsynaptic activity in an activity-dependent synaptic plasticity process.

15. The method of claim 9, wherein configuring a synaptic model utilizes a neural net.

16. The method of claim 9, further comprising instantiating the synaptic model, at least partially, in digital logic within the computation core.

17. A non-transitory computer readable storage medium having data stored therein representing software executable by a computer, the software including instructions that when executed cause the computer to perform the steps of:
configuring a synaptic model comprised by:
configuring a presynaptic component including at least a presynaptic target having, at least, a plasticity parameter and activity spike strength;
configuring a retrograde signaling component including a retrograde messenger; and
configuring a postsynaptic receptor component;
selectively generating a molecular uptake signal from the retrograde messenger;
modeling a presynaptic target at the retrograde messenger by modulating the plasticity parameter and activity spike strength of the presynaptic component based upon a calculated molecular uptake at the postsynaptic receptor component; and
transmitting the molecular uptake signal to the presynaptic component.

18. The computer readable storage medium of claim 17, wherein the software further causes the computer to perform the steps of:
modulating a molecular uptake signal of the retrograde signaling component which is modulatable in both duration and latency;
determining, at the retrograde signaling component, an uptake and degradation of molecules at the postsynaptic receptor component; and
wherein modulating the molecular uptake signal is based upon such determination.

19. The computer readable storage medium of claim 17, wherein the software further causes the computer to perform the step of losslessly transmitting the molecular uptake signal to the presynaptic component from the retrograde messenger.

20. The computer readable storage medium of claim 17, wherein the software further causes the computer to perform the steps of modeling one or both of ionotropic glutamate receptors (iGluR) and metabotropic glutamate receptors (mGluR).
